(19) United States
(12) Patent Application Publication
(10) Pub. No.: US 2010/0010354 A1
(43) Pub. Date: Jan. 14, 2010

(54) IMPLANTABLE ULTRASONIC MEASUREMENT ARRANGEMENT

(76) Inventors: Olaf Skerl, Bad Doberan (DE); Michael Lippert, Ansbach (DE); Gerald Czygan, Buckenhof (DE)

Correspondence Address: Intellectual Property Dept.;Dewitt Ross & Stevens SC, 2 East Mifflin Street, Suite 600, Madison, WI, 53703-2865, US

(21) Appl. No.: 12/497,778

(22) Filed: Jul. 6, 2009

(30) Foreign Application Priority Data
Jul. 9, 2008 (DE) DE 102008040266.4

(51) Int. Cl. A61B 8/14

(52) U.S. Cl. 600/459

(57) ABSTRACT

An implantable measurement arrangement for intracorporeal acoustic measurement of geometric parameters and motion parameters in and on organs and/or tissues of a patient includes an implantable device, in particular an electromedical device; an implantable sonic transducer for transmitting and receiving ultrasonic waves, the transducer being in signal connection with the implantable device; and an implantable reflector in communication with the implantable device and situated at a distance from the sonic transducer for reflecting the ultrasonic waves back in the direction of the sonic transducer. The electromedical device can analyze the ultrasonic waves picked up and reflected back by the sonic transducer.

### FIELD OF THE INVENTION

The invention relates to an implantable measurement arrangement for intracorporeal acoustic measurement of geometric parameters and/or motion parameters in and on organs/tissues of a patient. It also relates to an arrangement for remote monitoring of measured physiological variables of a patient based on such a measurement arrangement.

### BACKGROUND OF THE INVENTION

Ultrasonic medical diagnostic methods are standard, with ultrasonic imaging methods being among the most commonly used methods of testing a wide variety of organs. By using imaging methods, it is possible to determine geometric parameters such as size and position in relation to other organs. On the basis of the echo amplitude in certain areas, it is also possible to obtain information about the tissue structure in these areas.

Especially in cardiology today, ultrasonic techniques are some of the most important diagnostic methods. Transit time methods and imaging methods (echocardiography) are used to determine the geometric parameters of the heart. Doppler methods (Doppler sonography) can provide information about blood flow, and the movements of the cardiac walls can be examined with so-called tissue Doppler. In echocardiography, the dimensions of the left ventricle (LV) can be determined during the systole and the diastole, thus allowing determination of the stroke volume (SV), among other parameters.

In Doppler methods, the velocity of echogenic structures is determined based on the frequency shift of the echo (Doppler effect). This firstly makes it possible to measure the blood flow rate in a certain area (flow Doppler). In addition, the velocity of movement of organs or organ parts, e.g., the movement of the cardiac walls, can be measured by a noncontact method (tissue Doppler).

The two Doppler methods are standard diagnostic procedures today. For example, parameters for assessing cardiac hemodynamics can be obtained from the time characteristic of the blood flow rate in the LV outflow tract determined with the help of the flow Doppler method. With tissue Doppler, parameters for diagnosis of the contraction performance and contraction dynamics of the heart can be determined, for example. The parameters determined by means of ultrasonic methods are considered today as generally accepted clinically and are also frequently used as reference parameters. These methods are often used in combination. In recent years, intracardiac ultrasonic measurements have become increasingly important because they permit much more direct examination of the heart. It would therefore be useful to utilize these ultrasonic measurements in an implantable medical device (IMD), e.g., in a heart pacemaker or ICD for diagnostic purposes or for optimization of treatment.

All ultrasonic methods use ultrasonic waves that are reflected or backscattered into the tissue by inhomogeneities in the acoustic impedance. The reflection coefficient (R), i.e., the ratio of the reflected sonic energy to the incident sonic energy increases as the difference in the respective acoustic impedances Zₐᵢ and Zₐ₂ increases:

$$R = \frac{{( {Z}_{a 1} - {Z}_{a 2} )}^{2}}{{( {Z}_{a 1} + {Z}_{a 2} )}^{2}}$$

Since the differences in acoustic impedances between the various body tissues are generally minor, only a small fraction of the incident energy is received as a useful signal (echo). Detection of the received signal and its processing therefore require a high technical expenditure, and classification of the echo is usually possible only by consulting a skilled operator (usually a physician).

After transmitting the ultrasonic pulse, a plurality of echoes are received from different distances and different angles with a great variation in intensity. The receiving signal consequently contains all these echoes superimposed. With the help of technical means, the distance and angle range from which the received echoes are taken into account may optionally be limited. With the usual ultrasonic diagnostic methods, the sonic transducer is manually positioned by a skilled operator and aligned with the region to be examined. The measurement positions are also selected and marked manually on the basis of the assessment of results (e.g., ultrasonic images) by the skilled operator. Complex technical measures today can support the operator in these activities. Automatic selection of the echoes relevant for the measurement position is not generally possible.

With some applications, commercially available ultrasonic contrast media are used to improve the quality of the received signals. Ultrasonic contrast media contain small gas bubbles, for example, which have a high difference in acoustic impedance in relation to the environment and thus backscatter a large portion of the sonic energy. Tissues or organs marked in this way can therefore be differentiated clearly from the environment. For example, this allows imaging of blood vessels that would not be visible in the ultrasonic image without the contrast medium.

For some time now, there have also been efforts to equip pacemaker electrodes and catheters with ultrasonic marking in addition to the X-ray marking that has already become standard. Such markings can assist with reliable visibility of the electrode and/or catheter or their marked areas in the ultrasonic images. Ultrasonic imaging methods may thus be used as an alternative to X-rays, e.g. in implantation or follow-up care, and thus may reduce the radiation burden to the patient.

In general, the ultrasonic diagnostic methods may be performed noninvasively by applying a sonic transducer to the body from the outside (e.g., transthoracically) as well as invasively, for example, by inserting an ultrasonic catheter into the blood vessels (e.g., IVUS, intravascular ultrasound). One problem with all ultrasonic diagnostic methods is that the energy backscattered by the tissue structures is very low. Therefore, the echoes are generally vague and are superimposed on noise and interference signals (see FIG. 1A). A high signal processing effort is required to detect the individual echoes and isolate them from one another.

An accurate alignment of the sound beam with the region to be tested is also extremely important. In general, the narrowest possible sound beam is the goal. This ensures that the received signal will contain mainly information from the desired region and that the echoes can be allocated to certain structures. However, accurate positioning and alignment of the sonic transducer has proven to be extremely complex in the case of implants. The long-term stability of this positioning is also very difficult to ensure.

Known methods for using ultrasound in IMDs, such as those described in U.S. Pat. No. 5,544,656 or U.S. Pat. No. 6,421,565, have disadvantages. For example, if the alignment of the sonic transducer changes, it is no longer certain that the echoes received originate from the desired region. On the basis of the signals, this change can be detected only under certain conditions and with great technical effort.

To solve these problems, there are known arrangements that use at least two separate sonic transducers, one sonic transducer operating as a transmitter and the second sonic transducer (as well as any others) operating as a receiver (see U.S. Pat. No. 6,795,732 or US 2005/0027323, for example). This arrangement is often referred to as a microsonometer. The sonic transducers are positioned so that the desired diagnostic task is fulfilled. The advantage here is that the receiving transducer receives the directly incoming sound. This signal can be isolated clearly from the interference signals by simple means. Since the sound is sent by only one sonic transducer and is received directly, the assignment of the received signals to their source and the propagation path of the sound wave are unambiguous.

It is thus possible to use sonic transducers having a very broad transmission and/or reception angle, so that the requirements regarding accurate alignment and long-term stability need not be very high. The disadvantage of this arrangement is that it requires at least two sonic transducers with their feeder lines. The feeder lines in particular often pose a problem with long-term implants and limit the implantation sites.

In addition, electrodes and catheters have recently also been provided with additional ultrasonic markings to improve their visibility in the ultrasonic images (see, e.g., U.S. Pat. No. 4,805,628; U.S. Pat. No. 5,383,466; U.S. Pat. No. 5,921,933; U.S. Pat. No. 6,506,156; and U.S. Pat. No. 7,014,610). For example, small gas bubbles may be enclosed in areas of the sheathing. Likewise, implantable stents with ultrasonic markings are known (e.g., U.S. Pat. No. 5,289,831 and WO 2004/0103207). Due to the great difference in the acoustic impedance of the gas bubbles in comparison with the environment, there is a great reflection of the sonic waves on them. These ultrasonic markings therefore supply strong echoes and are thus also suitable as a reflection target for other intracorporeal ultrasonic measurements.

To be able to use ultrasonic diagnostic methods in long-term implants, with their limited resources of power and signal processing capacity, requires sharp and easily detectable echoes of structures in positions that are stable and precisely known.

### BACKGROUND OF THE INVENTION

The object of the present invention is therefore to provide a suitable measurement arrangement which is also inexpensive and easy to handle in clinical use. This object can be achieved by a measurement arrangement having the features set forth in the accompanying claims.

The invention is based on the idea of an intracorporeal acoustic measurement of geometric parameters and motion parameters in and on organs with the help of ultrasonic reflectors placed in defined positions. In this way, lower demands may be made of the positioning and alignment of the ultrasonic transducers than with the known arrangements. Ultrasonic transducers with a broad or spherical directional characteristic may be used so that changes in alignment have only a minor effect on signal quality. The effort for signal processing is reduced in comparison with that of known arrangements to such an extent that the received signals can be analyzed with the limited resources of a long-term implant. Another advantage of the inventive approach is that, in contrast with the known arrangements, at most only one sonic transducer with the respective feeder line is necessary.

Implanted bodies made of an ultrasound-reflecting material fixed at defined points in the body tissue are used as ultrasonic reflectors. Likewise, electrodes or stents with ultrasonic marking may also be used, for example. These reflectors supply significant echoes that can be isolated from the interference signals with little effort (see FIG. 1B). In addition, the position of these reflectors is known or can be ascertained by customary methods.

The distinctive echoes can be extracted from the received signal by a simple evaluation of the amplitude, for example. In addition, it is also possible to limit the signal range for detection of the echo by using a time window. This arrangement is not limited to ultrasonic pulse methods but may also be used for continuous methods (CW ultrasound) and in this case the sonic transducer contains a transmitting part and a receiving part.

In pulsed methods, the sonic transducer may also be switched from transmitting mode to receiving mode. With this arrangement, the transit time of sound from the transmission point in time until the arrival of the echo, the signal amplitude of the echo and the frequency shift (Doppler frequency) of the echo or all these parameters simultaneously can be determined with methods that are generally known. The respective distance between the ultrasonic transducer and the reflector can be determined from the transit time of the sound and this makes it possible to diagnose geometric changes in the section of organ under observation, for example. With a constant distance between the sonic transducer and the reflector, the velocity of sound in the observed section can be determined from the transit time of the sound, which can be used to derive statements about changes in the properties of the tissue in between, for example. Tissue damping can be determined from the signal amplitude of the echo, which also provides information about the properties of the tissue.

The velocity of the reflector in terms of amount and direction relative to the sonic transducer can be determined on the basis of the Doppler frequency of the received echoes. If the reflector is securely attached to the organ section to be tested, the velocity of the moving organ section is obtained and thus information corresponding to that of tissue Doppler is obtained.

Using the inventive arrangement, it is possible with the limited resources of an implantable medical device (IMD) to determine a number of parameters that are known and widely used in echography or in tissue Doppler analyses. The IMD may serve here as an implant with a therapeutic function, or may be designed solely as a monitoring implant for monitoring the patient's health condition.

For example, piezoelectric ceramics, piezoelectric polymer films or capacitive micromachined ultrasonic transducers (CMUT) may be used as the sonic transducers.

The ultrasonic measurement may be performed continuously, in a suitable timeframe or intermittently at defined points in time or at defined intervals. The measurements may also be synchronized with sensors for other physiological parameters, or with physiological events, or may be triggered by them.

Suitable parameters can be determined with the data obtained from the ultrasonic measurement. Determination of the parameters may be performed in the IMD itself or in an external device that is connected to the IMD in a suitable manner via a telemetry connection. The IMD may also transmit the determined parameters to the external device via the suitable telemetry connection.

The data obtained or the parameters determined from the data may be used for pure diagnostic purposes or for optimization of the therapeutic parameters of the IMD, whereby the optimization of the therapeutic parameters may also be performed in a closed loop. The optimization of the therapeutic parameters may be performed directly in the IMD or by using the external device connected to the IMD by telemetry connection. Furthermore, the data thereby obtained, the parameters determined from the data, or both may be stored in the IMD or the external device for a suitable period of time to make them available at a suitable point in time. Furthermore, trends or trend parameters, which can be compared with fixed threshold values, for example, to generate alarm messages, may also be determined from the data thereby obtained, from the parameters, or from a combination of the two. The determination of the trends, the comparison with the threshold values, and the generation of the alarms may be performed completely or partially in the IMD or completely or partially in the external device.

### BRIEF DESCRIPTION OF THE DRAWINGS

Advantages and features of the invention will also be apparent from the following description of exemplary versions in association with the accompanying drawings, wherein:

FIGS. 1A and 1B show graphic plots of ultrasonic signals obtained in the body of a patient, whereby FIG. 1A shows a typical ultrasonic echo signal which is subject to interference, and FIG. 1B shows an ultrasonic echo signal obtained by use of a reflector in one version of the invention;

FIG. 2 shows an overall view of a first exemplary version of the invention;

FIG. 3 shows an overall view of a second exemplary version of the invention, and

FIG. 4 shows a block diagram illustrating the signal processing in another version of the invention.

### DETAILED DESCRIPTION OF PREFERRED VERSIONS OF THE INVENTION

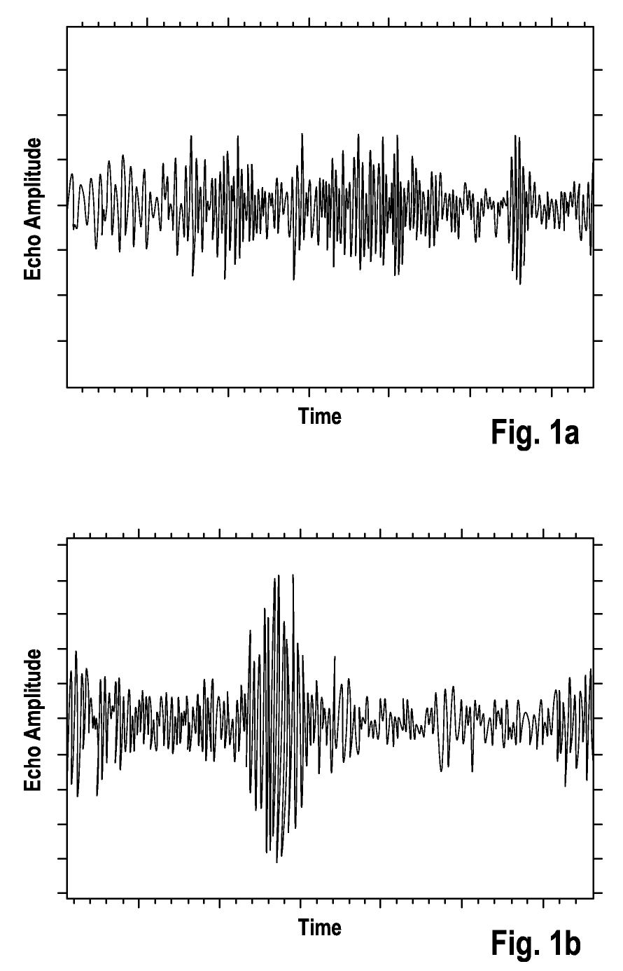

FIGS. 1A and 1B illustrate possible improvements in signal quality available by use of the invention in comparison with the known art.

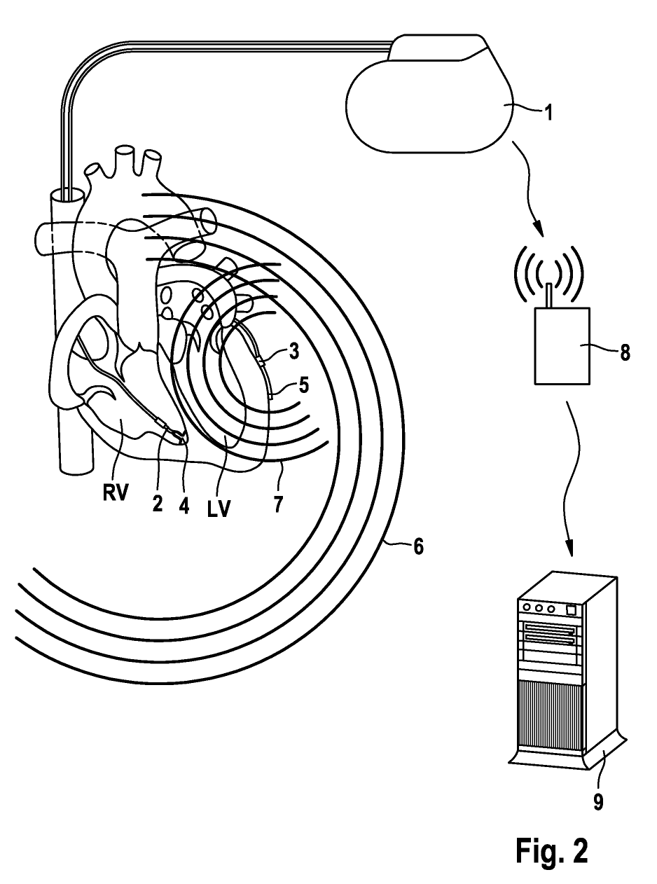

FIG. 2 shows an IMD 1 designed as a heart pacemaker or an ICD, having two electrodes 2, 3, one electrode 2 being placed in the right ventricle RV (RV electrode) and the second electrode 3 being placed in the coronary sinus on the left ventricle LV (LV electrode). The RV electrode 2 also contains a sonic transducer 4 for transmitting and receiving of ultrasonic pulses, as well as containing the required electric feeder lines and connections (not shown separately here). The sonic transducer 4 has a broad directional characteristic, so that only minor requirements are made of its alignment relative to the reflector. The LV electrode 3 additionally contains suitable ultrasonic markings 5 on its distal end which are used as reflectors. Instead of the LV electrode 3 with ultrasonic markings 5, a stent with ultrasonic marking in the coronary sinus may be placed in the coronary sinus or another suitable location on the LV. In addition to the usual electronic devices that are essential for functioning, The IMD also contains units for generating and receiving ultrasonic pulses and for analyzing the echoes received.

The IMD 1 sends an ultrasonic pulse 6 at a frequency of 1 to 5 MHz, for example, and a period of 1 to 10 μs, for example, via the sonic transducer 4 in the RV electrode 2. This sonic pulse 6 is backscattered on tissue structures within and in the vicinity of the heart and on the ultrasonic markings 5 of the LV electrode 3, which act as ultrasonic reflectors.

The energy backscattered by the ultrasonic markings 5 is many times greater than the energy of the tissue structures so that the echo 7 of the ultrasonic markings 5 is definitely emphasized in the received signal and can be isolated from the interference signals, for example, by a simple comparison with an amplitude threshold value (cf. FIG. 1B in this regard). In addition, interference signals can be masked out by using a suitable time window and only taking into account the echoes within this time window (for example, only between 30 and 150 μs after transmission of the sonic pulse). The transit time of the sound is determined based on the time between the transmission of the sonic pulse and the reception of the echo 7 of the ultrasonic marking 5 by using generally known methods. The distance between the sonic transducer 4 and the ultrasonic marking 5 is determined from this transit time of the sound.

Since both the sonic transducer 4 and the ultrasonic marking 5 are situated at precisely known locations in and/or on the heart, a measure of the extent of the LV can be obtained from the distance between the two. If the distance measurement described here is performed at a rate of 100 measurements per second, for example, the change in size of the LV is obtained on the basis of the heart cycle. Suitable parameters, e.g., the end diastolic and end systolic intervals can be determined from the change in size of the LV, allowing determination of the stroke volume.

Determination of the parameters may be performed directly in the IMD. The IMD may thus automatically adjust suitable therapeutic parameters, e.g., the AV or VV delay time or the efficacy of stimulation. To conserve energy, this measurement may be performed only once an hour for 20 seconds, for example. The distance data or the parameters determined from the data may be stored in the IMD and may be transmitted via a telemetry connection to a patient device 8 and a home monitoring service center 9. For example, trend parameters, which can be tied into an early warning system for exacerbation of the cardiac insufficiency, can be determined from these parameters. The parameters or trend parameters may also be compared with a threshold value to generate an alarm message for the patient or for the treating physician when there is a critical exacerbation of health status.

Another version of the invention, shown in FIG. 2, includes an IMD 1 and an electrode arrangement 2, 3 like that in FIG. 1. Unlike the situation described above, however, the transit time of the echoes is not analyzed but instead their frequency shift is analyzed based on the Doppler effect. If the ultrasonic marking 5 on the LV electrode moves relative to the sonic transducer 4, then the echoes backscattered by it will have a frequency shift in comparison with the frequency of the transmitted ultrasonic pulse, such that the frequency shift is proportional to the relative velocity between the sonic transducer and the ultrasonic marking (Doppler effect).

The echo of the ultrasonic marking may in turn be isolated from the interference signals by a simple amplitude assessment. A time window may also be used. The frequency shift (Doppler frequency) is then determined by the IMD from the echo of the ultrasonic marking 5 by using methods that are generally known. The ultrasonic marking 5 is attached to the wall of the LV by securing the LV electrode 3, so the myocardial velocity of the wall of the LV relative to the sonic transducer can be determined by a method similar to that in the generally known tissue Doppler measurement. If this velocity measurement is performed at a rate of 100 measurements per second, for example, this yields the characteristic of the myocardial velocity of the LV during the heart cycle. Parameters that describe the kinetics of the heart contraction can be determined from this velocity characteristic. The determination of the parameters may be performed in the IMD or in an external device.

The maximum myocardial velocity during a systole, which is known from tissue Doppler diagnostics, may be used as such a parameter, for example. On the basis of these parameters, the therapeutic parameters of the IMD can be optimized or balanced. Chronological relationships between the intracardiac electrogram (IEGM) and/or an electric stimulus and the myocardial movement may also be determined as parameters. For example, the LV reaction time can be determined from the interval of time between the electric stimulus on the LV electrode and the maximum of the myocardial velocity of the LV during the systole. For example, it is thus possible for the IMD to automatically optimize and adjust the ventricle-to-ventricle delay in cardiac resynchronization therapy (CRT).

The rate characteristics (or the parameters determined from them) may in turn be stored in the IMD 1 in a suitable manner and transmitted to the external patient device 8 and a home monitoring service center 9 via a telemetry connection. Trend parameters which are used for monitoring of the course of the disease, for example, or which can be tied into an early warning system may be determined from these parameters. The parameters or trend parameters may also be compared with a threshold value to generate an alarm message for the patient or the treating physician in the event of a critical exacerbation of the patient's health status.

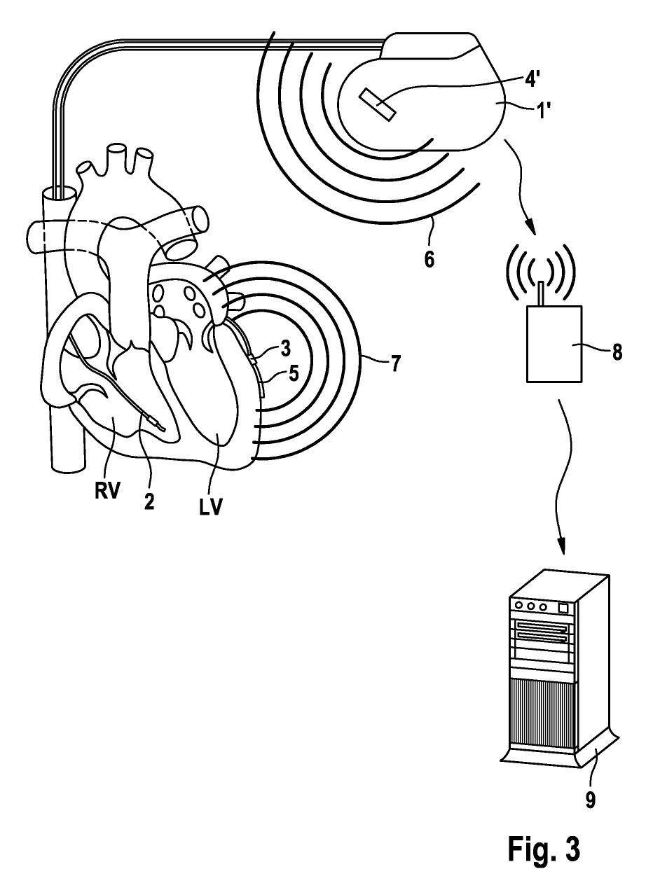

Another version illustrated in FIG. 3 contains an IMD 1′ provided in the form of a heart pacemaker and having an electrode arrangement similar to that in FIG. 2, with an RV electrode 2, and an LV electrode 3 which is placed in the coronary sinus on the LV (LV electrode) and is provided with an ultrasonic marking 5. Instead of an LV electrode with ultrasonic marking, another implant with an ultrasonic marking (e.g., a stent) may be placed at a suitable position in the coronary sinus. The IMD additionally contains at least one sonic transducer 4′ for transmitting and receiving ultrasonic waves, preferably in the direction of the ultrasonic marking on the LV. This arrangement has the advantage that a special electrode line containing a sonic transducer and special feeder lines are not required.

The IMD 1 sends a sonic pulse 6 via the sonic transducer 4′ contained in it and the sonic pulse propagates in the tissue. Due to the greater distance between the sonic transducer and the reflector, a lower sonic frequency, e.g., in the range between 100 kHz and 1 MHz, is more favorable because of the lower attenuation in the tissue here. Accordingly, an increased pulse length of 10 to 100 μs is recommended. This sonic pulse is backscattered at tissue structures and at ultrasonic marking 5 in the CS.

The energy reflected by the ultrasonic marking is in turn greater than the energy backscattered at the tissue structures so that the echo of the ultrasonic marking 7 in the received signal is definitely emphasized, and can be isolated from the interference signals by (for example) simply isolating signals above an appropriate amplitude threshold value. In addition, interference signals can be masked out by using a suitable time window by taking into account only the echoes within this time interval (for example, only between 100 and 300 μs after transmission of the sonic pulse). The Doppler shift of the echoes can be used as another criterion for isolation of the echoes of the ultrasonic marking from the interference signals. The echoes of the ultrasonic markings connected to the beating heart have the greatest Doppler shifts because the cardiac walls represent the fastest-moving structure in the chest cavity. All other structures that generate an echo signal, such as the pulmonary alveoli or the skin surface, move much more slowly and therefore have a much smaller Doppler shift and can be isolated from the echoes of the ultrasonic marking by using generally methods, e.g., by filtering.

The transit time of the sound is determined by using commonly known methods based on the time between the transmission of the sonic pulse and the reception of the echo of the ultrasonic marking. The distance between the sonic transducer and the IMD and the ultrasonic marking is determined from this transit time of the sound. If the distance measurement described here is performed at the rate of 100 measurements per second, for example, this yields information about the movement of the LV during the heart cycle. The Doppler shift of the echoes of the ultrasonic marking can also provide information about the velocity of the LV and the contraction dynamics of the LV. Suitable parameters in the IMD or in an external device may in turn be determined from the distance or rate characteristics in the IMD or in an external device.

The characteristics or the parameters determined therefrom can be used to optimize the therapeutic parameters of the IMD, stored in the IMD, and/or transmitted via a telemetry connection to an external patient device 8 and to a service center 9, and suitable trend parameters can be determined from them and can be used for monitoring the course of the disease, for example, or can be tied into an early warning system. The parameters or trend parameters may also be compared with a threshold value to generate an alarm message for the patient or the treating physician in the event of a critical exacerbation of the patient's health status.

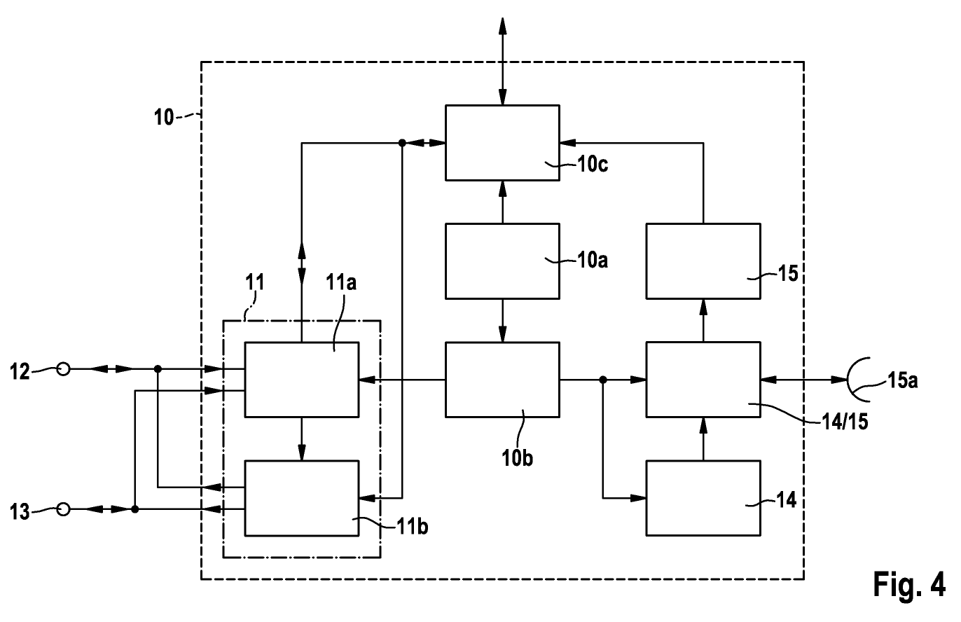

FIG. 4 shows schematically in a block diagram of preferred functional elements of an implantable electromedical device 10 in one version of the invention, which may be provided in an arrangement of the type shown in FIG. 3, for example. The device 10 comprises a pacemaker component 11 connected to sensing and stimulation electrodes 12, 13, an ultrasonic generator component 14 and an acoustic measurement component 15.

The pacemaker component 11 comprises a sensing part 11a and a stimulation part 11b, each being connected to the electrodes 12 (RV electrode) and 13 (LV electrode) in the embodiment illustrated here in order to detect heart action potentials in the respective ventricle and deliver stimulation pulses thereto, if necessary. The sensing part 11a is connected at the output to a control input of the stimulation part 11b in the usual manner.

A controller 10a controls the functions of the device 10 that are to be coordinated with one another, and specifically controls, via a synchronization step 10b, the synchronization in time between the detection processes of the sensing part 11a of the pacemaker 11 and the acoustic measurement component 15 that can be connected at the input, via a switching stage 14/15 at the input, to a sonic transducer 15a having a piezoelectric ceramic or a piezoelectric polymer film, for example. Operation of the ultrasonic generator 14, which can also be connected to the sonic transducer 15a via the switching stage 14/15, is also triggered by the synchronization stage 10b.

Finally, the controller 10a also controls the operation of a telemetry stage 10c, through which the sensing part 11a as well as the stimulation part 11b of the heart pacemaker and the acoustic measurement component 15 may be connected to an external patient device to supply measurement results of heart action potentials and acoustic measurements to the external patient device, and to obtain control commands for the heart pacemaker from the external patient device. The results of an external analysis of the measurements of the acoustic measurement component 15 may also be reflected in these control commands so that no internal feedback is needed in the pacemaker part. As an alternative, an analysis of the acoustic measurements internally within the device may also be provided, with the results then influencing the pacemaker operation. At least one of the electrodes 12, 13 then functions as the actuator of a pacemaker therapy.

The invention is not limited to the versions and examples presented here, and can implemented in a variety of different forms instead, with the various forms being encompassed by the claims below.

What is claimed is:

1. An implantable measurement arrangement for intracorporeal acoustic measurement of geometric and/or motion parameters of a patient's organs and/or tissue, including:
a. an implantable electromedical device;
b. an implantable sonic transducer in signal connection with the implantable electromedical device, the implantable sonic transducer being configured to transmit and receive ultrasonic waves; and
c. an implantable reflector situated at a distance from the sonic transducer for reflecting the ultrasonic waves in the direction of the sonic transducer,
wherein the implantable electromedical device is configured to analyze the reflected ultrasonic waves received by the sonic transducer.

2. The arrangement of claim 1 wherein the reflector is:
a. at least partially formed of ultrasonically reflecting material, and
b. fixed with respect to an organ and/or tissue of the patient.

3. The arrangement of claim 2 wherein the reflector is defined as at least a portion of a stent.

4. The arrangement of claim 1 wherein the sonic transducer includes an ultrasonic pulse generator generating ultrasonic pulses having a frequency between 0.1 and 5 MHz.

5. The arrangement of claim 1 wherein the ultrasonic transducer switches between:
a. a transmission mode wherein the ultrasonic transducer transmits ultrasonic waves, and
b. a receiving mode wherein the ultrasonic transducer receives reflected ultrasonic waves.

6. The arrangement of claim 1 wherein the sonic transducer includes one or more of:
a. a piezoelectric ceramic,
b. a piezoelectric polymer film, and
c. a capacitive micromachined ultrasonic transducer (CMUT).

7. The arrangement of claim 1 including an additional sensor detecting signals from an organ and/or tissue of the patient, the additional sensor being in communication with the implantable electromedical device and being situated on the organ and/or the tissue of the patient, wherein the implantable electromedical device synchronizes the sonic transducer's reception of the ultrasonic waves with detection of signals via the additional sensor.

8. The arrangement of claim 6 wherein the implantable electromedical device triggers the measurement of heart action potentials by the additional sensor.

9. The arrangement of claim 1 further including an electrode delivering stimulation pulses to an organ and/or tissue of the patient in response to the analyzed reflected ultrasonic waves.

10. The arrangement of claim 1 wherein:
a. the sonic transducer is affixed to the implantable electromedical device, and
b. the reflector is affixed to the organ and/or the tissue of the patient.

11. The arrangement of claim 1 wherein:
a. the implantable electromedical device is a heart pacemaker;
b. the sonic transducer at is at or adjacent to the distal end of an electrode line which is placed in or on a ventricle;
c. the reflector is at or adjacent to the distal end of an electrode line placed in or on the other ventricle.

12. The arrangement of claim 11 wherein at least one of:
a. the implantable electromedical device, and
b. an external device in wireless communication with the implantable electromedical device,
determine at least one of:
(1) the change in size of one or more of the ventricles, and
(2) the myocardial velocity,
based on the reflected ultrasonic waves received by the sonic transducer.

13. The arrangement of claim 1 further including:
a. an external device in wireless communication with the implantable electromedical device,
b. a remote processing unit in communication with the external device,
wherein:
(1) the external device wirelessly receives signals indicative of the reflected ultrasonic waves from the implantable electromedical device;
(2) the remote processing unit:
(a) generates control signals in response to the signals indicative of the reflected ultrasonic waves, and
(b) transmits the control signals to the implantable electromedical device,
with the control signals subsequently controlling the implantable electromedical device.

14. An implantable measurement arrangement for intracorporeal acoustic measurement of geometric and/or motion parameters of a patient's organs and/or tissue, including:
a. an implantable electromedical device;
b. an implantable sonic transducer in communication with the implantable electromedical device, the implantable sonic transducer being configured to:
(1) emit ultrasonic waves, and
(2) receive reflections of the emitted ultrasonic waves;
c. an implantable reflector situated at a distance from the sonic transducer for reflecting the ultrasonic waves,
d. an implantable electrode lead in communication with the implantable electromedical device, the electrode lead including:
(1) one of the implantable sonic transducer and the implantable reflector; and
(2) one or more of:
(a) a stimulation electrode for delivering an electrical stimulation pulse to the patient's organs and/or tissue adjacent to the stimulation electrode;
(b) a detection electrode for detecting electrical potentials in the patient's organs and/or tissue adjacent to the stimulation electrode.

15. The measurement arrangement of claim 14 wherein the other of the implantable sonic transducer and the implantable reflector not included on the electrode lead is situated on the implantable electromedical device.

16. The measurement arrangement of claim 14 wherein the other of the implantable sonic transducer and the implantable reflector not included on the electrode lead is situated on an electrode lead separate and spaced from the electrode lead on which the one of the implantable sonic transducer and the implantable reflector is included.

17. The measurement arrangement of claim 14 wherein electrical stimulation pulses are delivered to the patient's organs and/or tissue, with the delivery of the pulses being at least partially determined by the reflections of the emitted ultrasonic waves received at the implantable sonic transducer.

18. The measurement arrangement of claim 14 further including an external device in wireless communication with the implantable electromedical device, wherein the external device:
a. wirelessly receives signals representative of the ultrasonic waves reflected from the reflector, and
b. wirelessly transmits to the implantable electromedical device instructions for delivery of an electrical stimulation pulse to the patient's organs and/or tissue.

19. An implantable measurement arrangement for intracorporeal acoustic measurement of geometric and/or motion parameters of a patient's organs and/or tissue, including:
a. an implantable electromedical device;
b. an implantable sonic transducer in communication with the implantable electromedical device, the implantable sonic transducer being configured to emit ultrasonic waves;
c. an implantable electrode lead in communication with the implantable electromedical device, the electrode lead including:
(1) an implantable sonic reflector spaced from the sonic transducer, and
(2) one or more of:
(a) a stimulation electrode for delivering an electrical stimulation pulse to the patient's organs and/or tissue adjacent to the stimulation electrode;
(b) a detection electrode for detecting electrical potentials in the patient's organs and/or tissue adjacent to the stimulation electrode.

20. The measurement arrangement of claim 19 wherein the implantable sonic transducer is situated on one of:
a. a second electrode lead separate and spaced from the electrode lead; and
b. the implantable electromedical device.
